(19) United States
(12) Patent Application Publication
(10) Pub. No.: US 2009/0104126 A1
(43) Pub. Date: Apr. 23, 2009

(54) Sulfamate Compounds and Uses Thereof

(75) Inventors: Uwe SCHOEN, Hannover (DE); Harald Waldeck, Hannover (DE); Uwe Reinecker, Hannover (DE); Peter-Colin Gregory, Hannover (DE); Dania Reiche, Hannover (DE); Holger Sann, Hannover (DE); Michael Wurl, Hannover (DE); Jochen Antel, Hannover (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 12/253,730

(22) Filed: Oct. 17, 2008

Related U.S. Application Data
(60) Provisional application No. 60/981,326, filed on Oct. 19, 2007.

(51) Int. Cl. A61K 9/12; C07C 307/02; A61K 31/255

(52) U.S. Cl. 424/45; 558/48; 514/511

(57) ABSTRACT

A sulfamate compound corresponding to Formula I

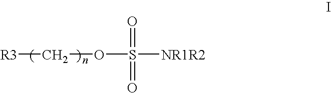

wherein R1 to R3 and n have defined meanings, pharmaceutical compositions comprising these compounds; a process for preparing these compounds, and the use of such compounds or compositions to treat or inhibit various disorders or disease states in patients in need thereof by administering to such a patient a therapeutically effective amount of such a compound.

### FIELD OF THE INVENTION

This invention relates to the fields of pharmaceutical and organic chemistry, and provides new sulfamate compounds, medicaments comprising these compounds, pharmaceutical compositions comprising these compounds, and processes for the preparation of these compounds. The invention also concerns the uses of such compounds and compositions, particularly their use in administering them to patients to achieve a therapeutic effect.

### BACKGROUND OF THE INVENTION

The use of sulfamates as pharmaceutical actives in medicine is known for many years. EP 0 138 441 discloses sulfamate derivatives of the following formula

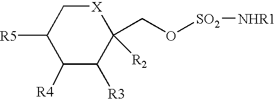

wherein X is O or CH2, R1 is hydrogen or alkyl, R2 to R5 are hydrogen or alkyl, and when X is CH2, R4 and R5 may be joined to form a benzene ring, and, when X is O, R2 and R3 and/or R4 and R5 together may be a methylenedioxy group. Compounds of the described type have been reported to exhibit anticonvulsant properties. EP 0 138441 further describes the use of these compounds in the treatment of diseases such as epilepsy and glaucoma.

Maryanoff et al. disclose in J. Med. Chem. 1998, 41, 1315 to 1343 additional sulfamate derivatives for use in medical chemistry. It is reported that beta-D-fructopyranose sulfamates exhibit anticonvulsant activities analogously to that of phenyloin. Topiramate of formula below is a known representative, studied for the treatment of a variety of medicial conditions such as epilepsy in both children and adults.

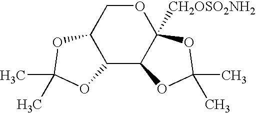

In children, Topiramate is also indicated for treatment of Lennox-Gastaut syndrome (a disorder that causes seizures and developmental delays). Topiramate is also Food and Drug Administration (FDA) approved for, and now most frequently prescribed for the prevention of migraines. Topiramate has been used by psychiatrists to treat bipolar disorder although it is not FDA approved for this purpose. This drug has been investigated for use in treatment of obesity especially to aid in the reduction of binge eating, and also as a possible treatment for alcoholism. The drug is also used in clinical trials to treat post traumatic stress disorder. A pilot study suggests that Topiramate is possibly effective against infantile spasm. In May 2006 the U.S. National Institutes of Health web site http://www.clinicaltrials.gov listed several studies sponsored by Ortho-McNeil which propose to examine the use of Topiramate on migraine, cluster and severe headaches within various demographics. Other off-label and investigational uses of Topiramate include: treatment of bulimia nervosa, obsessive-compulsive disorder, smoking cessation, and treatment of neuropathic pain.

Maryanoff et al further disclose in J. Med. Chem. 1987, 41, 880 to 887 two bicyclic sulfamates.

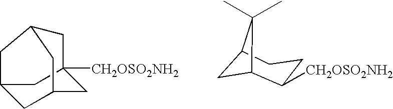

Their activity as anticonvulsants is however reported to be low.

### SUMMARY OF THE INVENTION

It was an object of the present invention to provide novel sulfamates, which are very effective and can be obtained in simple manner, for the treatment and/or prophylaxis of various medical conditions.

It has now surprisingly been found that certain novel sulfamates and their physiologically acceptable salts, hydrates and solvates are suitable for the treatment and/or prophylaxis of various diseases or conditions, such as obesity, diabetes mellitus type I, diabetes mellitus type II, metabolic syndrome, syndrome X, diabetic neuropathy, diabetic retinopathy, diabetic nephropathy, diabetic microangiopathy, diabetic macroangiopathy, insulinoma, familial hyperinsulemic hypoglycemia, male pattern baldness, detrusor hyperreactivity, hypertension, in particular arterial hypertension, dyslipoproteinaemia, in particular as hypertriglyceridaemia accompanied by dyslipoproteinaemia occurring with/without lowered HDL-cholesterol; hyperuricaemia; asthma, glucose metabolism, in particular insulin resistance, hyperglycaemea and/or glucose intolerance, neuroprotection, Parkinson Disease, Alzheimer Disease, analgesia, angina, arrhythmia, coronary spasm, peripheral vascular disease, cerebral vasospasm, appetite regulation, neurodegeneration, pain, including neuropathic pain and chronic pain, impotence, glaucoma, bipolar disorders, migraine, alcohol dependence, cancer and cardiovascular disease, which comprises in particular cardioprotection, cardioplegia, coronary heart disease, cerebrovascular diseases and peripheral occlusive arterial disease and their concomitant and/or secondary diseases or conditions.

It has been found that compounds of Formula I are new and are suitable for the treatment of various medical conditions. This invention relates to compounds of Formula I

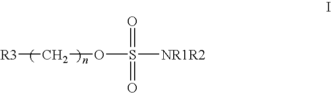

### Wherein

R1 and R2 are independently selected from the group consisting of: hydrogen, C₁ to C₈ alkyl, C₄ to C₁₀ cycloalkyl, aryl and heteroaryl, of which alkyl and cycloalkyl are optionally substituted with at least one substitutent Y and of which aryl and heteroaryl are optionally substituted with at least one substitutent Z, or wherein R1 and R2 form together a 5 or 6-membered ring which may additionally contain from 1 to 2 heteroatoms independently selected from the group consisting of: nitrogen, oxygen and sulphur and which 5 or 6-membered ring is optionally substituted with at least one substituent Y;
R3 is selected from the group consisting of: (1S,2S,5S)-6,6-dimethyl-bicyclo[3.1.1]hept-2-yl; (1R,2R,5R)-6,6-dimethylbicyclo[3.1.1]hept-2-yl; (1S,2R,5S)-6,6-dimethyl-bicyclo-[3.1.1]hept-2-yl; (1R,4S)-bicyclo[2.2.1]hept-2-yl; (1S,4R)-3-methyl-bicyclo[2.2.1]hept-2-yl; bicyclo[2.2.2]oct-5-en-2-yl; (4S)-bicyclo[2.2.1]hept-5-en-2-yl; (1S,2R,4S)-1,7,7-trimethyl-bicyclo[2.2.1]hept-2-yl; (1R,2S,4R)-1,7,7-trimethyl-bicyclo[2.2.1]hept-2-yl; and (1R,2R,4R)-1,7,7-trimethylbicyclo[2.2.1]hept-2-yl;
n is an integer from 0 to 3;
Y is selected from the group consisting of: alkyl, alkoxy, thioalkyl, aryl, CO-aryl, heteroaryl, amino and carboxylalkyl; and
Z is selected from the group consisting of: alkyl, alkoxy, thioalkyl, halogen, aryl, CO-aryl, CN, heteroaryl and carboxylalkyl;
   and its physiologically acceptable salts, hydrates and solvates.

The invention further relates to pharmaceutical compositions comprising compounds of Formula I, to pharmaceutical compositions comprising compounds of Formula I, and processes for the preparation of compounds of Formula I. The invention also concerns the uses of compounds of Formula I and of compositions comprising compounds of Formula I, particularly their use in administering them to patients to achieve a therapeutic effect.

### DETAILED DESCRIPTION OF THE INVENTION

The invention particularly relates to compounds of Formula I wherein R1 and R2 are independently selected from the group consisting of: hydrogen and C₁ to C₈ alkyl, wherein C₁ to C₈ alkyl are optionally substituted with at least one substituent Y selected from the group consisting of: C₁-C₄ alkyl, C₁-C₄ alkoxy, C₁-C₄ thioalkyl, C₆-C₁₂ aryl, CO—C₆-C₁₂ aryl, C₆-C₁₂heteroaryl, amino, and carboxyl-C₁-C₄-alkyl.

Preferred are compounds of Formula I wherein R1 and R2 are both hydrogen.

In another embodiment of the present invention, compounds are preferred wherein n is 1 or 2, more preferably n is 1.

In another embodiment of the present invention, the compound is selected from the group consisting of [(1S,2S,5S)-6,6-dimethylbicyclo[3.1.1]hept-2-yl]methylsulfamate, [(1R,2R,5R)-6,6-dimethylbicyclo[3.1.1]hept-2-yl]methylsulfamate, [(1S,2S,5S)-6,6-di-methylbicyclo[3.1.1]hept-2-yl]ethylsulfamate, [(1S,2R,5S)-6,6-dimethylbicyclo[3.1.1]hept-2-yl]methylsulfamate, and (1R,4S)-bicyclo-[2.2.1]hept-2-yl-methylsulfamate, and (4S)-bicyclo[2.2.1]hept-5-en-2-ylmethylsulfamate. The most preferred compound of the present invention is [(1S,2S,5S)-6,6-dimethylbicyclo[3.1.1]hept-2-yl]methylsulfamate.

In another embodiment, the invention relates to a pharmaceutical composition comprising a compound of Formula I, or a pharmacologically acceptable salt, hydrate or solvate thereof and at least one pharmaceutically acceptable carrier or adjuvant.

In another embodiment, the present invention relates to a pharmaceutical composition comprising:
A) a pharmacologically effective amount of a compound of Formula I or a pharmacologically acceptable salt, hydrate or solvate thereof, as an active ingredient; and;
B) optionally at least one pharmaceutically acceptable carrier and/or at least one pharmaceutically acceptable auxiliary substance.

In another embodiment, the present invention relates to the use of a pharmaceutical composition comprising:
A) a pharmacologically effective amount of a compound of Formula I or a pharmacologically acceptable salt, hydrate or solvate thereof, as an active ingredient; and;
B) optionally at least one pharmaceutically acceptable carrier and/or at least one pharmaceutically acceptable auxiliary substance. to treat obesity, diabetes mellitus type I, diabetes mellitus type II, metabolic syndrome, syndrome X, diabetic neuropathy, diabetic retinopathy, diabetic nephropathy, diabetic microangiopathy, diabetic macroangiopathy, insulinoma, familial hyperinsulemic hypoglycemia, male pattern baldness, detrusor hyperreactivity, hypertension, in particular arterial hypertension, dyslipoproteinaemia, in particular as hypertriglyceridaemia accompanied by dyslipoproteinaemia occurring with/without lowered HDL-cholesterol; hyperuricaemia; asthma, glucose metabolism, in particular insulin resistance, hyperglycaemea and/or glucose intolerance, neuroprotection, Parkinson Disease, Alzheimer Disease, analgesia, angina, arrhythmia, coronary spasm, peripheral vascular disease, cerebral vasospasm, appetite regulation, neurodegeneration, pain, including neuropathic pain and chronic pain, impotence, glaucoma, bipolar disorders, migraine, alcohol dependence, cancer and cardiovascular disease, which comprises in particular cardioprotection, cardioplegia, coronary heart disease, cerebrovascular diseases and peripheral occlusive arterial disease and their concomitant and/or secondary diseases or conditions.

The compounds of the invention of Formula I, as well as the pharmacologically acceptable salts, hydrates and solvates thereof, have carbonic anhydrase enzyme inhibitory activity. They are useful in treating disorders involving carbonic anhydrase enzymes, or treatable by manipulation of those enzymes. For instance in treatment and/or inhibition of various diseases or conditions, such as obesity, diabetes mellitus type I, diabetes mellitus type II, metabolic syndrome, syndrome X, diabetic neuropathy, diabetic retinopathy, diabetic nephropathy, diabetic microangiopathy, diabetic macroangiopathy, insulinoma, familial hyperinsulemic hypoglycemia, male pattern baldness, detrusor hyperreactivity, hypertension, in particular arterial hypertension, dyslipoproteinaemia, in particular as hypertriglyceridaemia accompanied by dyslipoproteinaemia occurring with/without lowered HDL-cholesterol; hyperuricaemia; asthma, glucose metabolism, in particular insulin resistance, hyperglycaemea and/or glucose intolerance, neuroprotection, Parkinson Disease, Alzheimer Disease, analgesia, angina, arrhythmia, coronary spasm, peripheral vascular disease, cerebral vasospasm, appetite regulation, neurodegeneration, pain, including neuropathic pain and chronic pain, impotence, glaucoma, bipolar disorders, migraine, alcohol dependence, cancer and cardiovascular disease, which comprises in particular cardioprotection, cardioplegia, coronary heart disease, cerebrovascular diseases and peripheral occlusive arterial disease and their concomitant and/or secondary diseases or conditions.

The compounds of the invention possess carbonic anhydrase inhibitory activity. The inhibiting activities of the compounds of the invention are readily demonstrated, for example, using one or more of the assays described herein or known in the art.

Isolation and purification of the compounds described herein can be achieved, if desired, by any suitable separation or purification procedure such as, for example, filtration, extraction, crystallization, column chromatography, thin-layer chromatography, thick-layer chromatography, preparative low or high-pressure liquid chromatography, or a combination of these procedures. Specific illustrations of suitable separation and isolation procedures can be taken from the preparations and examples. However, other equivalent separation or isolation procedures could, of course, also be used.

The compounds of the present invention may contain one or more asymmetric centers and thus occur as individual enantiomers, diastereomeric mixtures and individual diastereomers.

Depending on the nature of the various substituents, the molecule can have additional asymmetric centers. Each such asymmetric center will independently produce two optical isomers. The independent syntheses of these diastereomers, or their chromatographic separations, may be achieved as known in the art by appropriate modification of the methodology disclosed therein. Their absolute stereochemistry may be determined by the X-ray crystallography of crystalline products or crystalline intermediates, which are derivatized, if necessary, with a reagent containing an asymmetric center of known absolute configuration. Racemic mixtures of the compounds can be separated into the individual enantiomers by methods known in the art, such as the coupling of a racemic mixture of compounds to an enantiomerically pure compound to form a diastereomeric mixture, followed by separation of the individual diastereomers by standard methods, such as fractional crystallization or chromatography. The coupling often consists of the formation of salts using an enantiomerically pure acid or base. The diasteromeric derivatives may then be converted to the pure enantiomers by cleavage of the added chiral residue. The racemic mixture of the compounds can also be separated directly by methods known in the art, such as chromatographic methods utilizing chiral stationary phases. Alternatively, any enantiomer of a compound may be obtained by stereoselective synthesis using optically pure starting materials or reagents of known configuration by methods well-known in the art.

Some of the crystalline forms for the compounds may exist as polymorphs within the scope of the present invention, which are considered to be part of the invention. In addition, some of the compounds may form solvates with water (i.e. hydrates), or common organic solvents. Such solvates also fall within the scope of this invention.

Isotopically-labeled compound of Formula I or pharmaceutically acceptable salts thereof, including compounds of Formula I isotopically-labeled to be detectable by PET or SPECT, also fall within the scope of the invention. The same applies to compounds of formula (I) labeled with [¹³C]—, [¹⁴C]—, [³H]—, [¹⁸F]—, [¹²⁵I]— or other isotopically enriched atoms, suitable for receptor binding or metabolism studies.

### DEFINITIONS

General terms used in the description of compounds herein disclosed bear their usual meanings.

The term alkyl as used herein denotes a univalent saturated branched or straight hydrocarbon chain. Unless otherwise stated, such chains can contain from 1 to 18 carbon atoms. Representatives of such alkyl groups are methyl, ethyl, propyl, isopropyl, butyl, isobutyl, sec-butyl, tert-butyl, pentyl, isopentyl, neopentyl, tert-pentyl, hexyl, isohexyl, heptyl, octyl, nonyl, decyl, undecyl, dodecyl, tridecyl, tetradecyl, pentadecyl, hexadecyl, heptadecyl, octadecyl, and the like. In a preferred embodiment of the present invention, the alkyl group contains from 1 to 8 carbon atoms. The same carbon content applies to the parent term ‘alkane’, and to derivative terms such as ‘alkoxy’ and ‘thioalkyl’.

The term ‘aryl’ embraces monocyclic or fused bi- and polycyclic aromatic groups, including but not limited to phenyl, 1,2,3,4-tetrahydro-naphtyl, naphthyl, and azulenyl.

The term ‘heteroaryl’ embraces monocyclic or fused bi- and polycyclic aromatic ring systems in which one or more carbon atoms have been replaced by a heteroatom. The term ‘heteroaryl’ includes but is not limited to furyl, thienyl, pyrrolyl, oxazolyl, thiazolyl, imidazolyl, imidazo[2,1-b][1,3]thiazolyl, pyrazolyl, isoxazolyl, isothiazolyl, pyridyl, pyridazinyl, pyrimidinyl, pyrazinyl, 1,3,5-triazinyl, indazolyl, indolyl, indolizinyl, isoindolyl, benzo[b]furanyl, 1,2,3,4-tetrahydroisoquinolinyl, indanyl, indenyl, benzo[b]thienyl, 2,3-dihydro-1,4-benzodioxin-5-yl, benzimidazolyl, benzothiazolyl, benzo[1,2,5]thia-diazolyl, purinyl, quinolinyl, isoquinolinyl, phthalazinyl, quinazolinyl, quinoxalinyl, 1,8-naphthyridinyl, naphthyl, pteridinyl, azulenyl, and the like.

‘Halogen’ means chloro, fluoro, bromo or iodo.

‘Hetero’ as in ‘heteroalkyl, heteroaromatic’ etc. means containing one or more N, O or S atoms.

The term “substituted” means that the specified group or moiety bears one or more substituents. Where any group may carry multiple substituents, and a variety of possible substituents is provided, the substituents are independently selected, and need not to be the same. The term “unsubstituted” means that the specified group bears no substituents.

‘Optionally substituted’ means that the alkyl or cycloalkyl groups may or may not be further substituted by one or more groups Y, or, that the aryl group may or may not be further substituted by one or more groups Z.

‘Crystal form’ refers to various solid forms of the same compound, for example polymorphs, solvates and amorphous forms. ‘Polymorphs’ are crystal structures in which a compound can crystallize in different crystal packing arrangements, all of which have the same elemental composition. Polymorphism is a frequently occurring phenomenon, affected by several crystallization conditions such as temperature, level of supersaturation, the presence of impurities, polarity of solvent, rate of cooling. Different polymorphs usually have different X-ray diffraction patterns, solid state NMR spectra, infrared or Raman spectra, melting points, density, hardness, crystal shape, optical and electrical properties, stability, and solubility. Recrystallization solvent, rate of crystallization, storage temperature, and other factors may cause one crystal form to dominate. ‘Solvates’ are generally a crystal form that contains either stoichiometric or non-stoichiometric amounts of a solvent. Often, during the process of crystallization some compounds have a tendency to trap a fixed molar ratio of solvent molecules in the crystalline solid state, thus forming a solvate. When the solvate is water, ‘hydrates’ may be formed. The compounds of Formula I and pharmaceutically acceptable salts thereof may exist in the form of a hydrate or a solvate, and such a hydrate and solvate are also encompassed in the present invention. Examples thereof include 1/10 hydrate, ¼ hydrate, ½ hydrate, monohydrate, dihydrochloride ½ hydrate, dihydrochloride dihydrate, dihydrochloride 3/2 hydrate, and the like. ‘Amorphous’ forms are noncrystalline materials with no long range order, and generally do not give a distinctive powder X-ray diffraction pattern. Crystal forms in general have been described by Byrn (1995) and Martin (1995).

With reference to substituents, the term “independently” means that when more than one of such substituents are possible, they may be the same or different from each other.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Throughout the description and the claims of this specification the word “comprise” and variations of the word, such as “comprising” and “comprises” are “open” terms and are not intended to exclude the possible presence of other additives, components, integers or steps.

While it may be possible for the compounds of Formula I to be administered as the raw chemical, it is preferable to present them as a ‘pharmaceutical composition’. According to a further aspect, the present invention provides pharmaceutical compositions comprising a compound of Formula I, or a pharmaceutically acceptable salt, hydrate or solvate thereof, optionally together with one or more pharmaceutically acceptable carriers and/or at least one pharmaceutically acceptable auxiliary substance. The carrier(s) and auxiliary substance(s) must be ‘acceptable’ in the sense of being compatible with the other ingredients of the formulation and not deleterious to the recipient thereof.

The term “composition” as used herein encompasses a product comprising specified ingredients in predetermined amounts or proportions, as well as any product that results, directly or indirectly, from combining specified ingredients in specified amounts. In relation to pharmaceutical compositions, this term encompasses a product comprising one or more active ingredients, and an optional carrier and/or auxiliary substances comprising inert ingredients, as well as any product that results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. The pharmaceutical composition includes enough of the active object compound to produce the desired effect upon the progress or condition of diseases. Accordingly, the pharmaceutical compositions of the present invention encompass any composition made by admixing a compound of the present invention and optionally a pharmaceutically acceptable carrier and/or auxiliary substance. By “pharmaceutically acceptable” it is meant the carrier, diluent or excipient must be compatible with the other ingredients of the formulation and not deleterious to the recipient thereof.

The affinity of the compounds of the invention as inhibitors of carbonic anhydrases was determined as described below. From the potency measured for a given compound of Formula I, one can estimate a theoretical lowest effective dose. At a concentration of the compound equal to twice the measured inhibition constant, nearly 100% of the carbonic anhydrase enzyme likely will be occupied by the compound. By converting that concentration to mg of compound per kg of patient one obtains a theoretical lowest effective dose, assuming ideal bioavailability. Pharmacokinetic, pharmacodynamic, and other considerations may alter the dose actually administered to a higher or lower value. The typical daily dose of the active ingredients varies within a wide range and will depend on various factors such as the relevant indication, the route of administration, the age, weight and sex of the patient, and may be determined by a physician. In general, total daily dose administration to a patient in single or individual doses, may be in amounts, for example, from 0.001 to 10 mg/kg body weight daily, of total active ingredients of Formula I. Such dosages will be administered to a patient in need of treatment from one to three times each day, or as often as needed for efficacy, and for periods of at least two months, more typically for at least six months, or chronically.

The term “therapeutically effective amount” as used herein refers to an amount of a therapeutic agent to treat a condition treatable by administrating a composition of the invention. That amount is the amount sufficient to exhibit a detectable therapeutic or ameliorative response in a tissue system, animal or human. The effect may include, for example, treating the conditions listed herein. The precise effective amount for a subject will depend upon the subject's size and health, the nature and extent of the condition being treated, recommendations of the treating physician (researcher, veterinarian, medical doctor or other clinician), and the therapeutics, or combination of therapeutics, selected for administration. Thus, it is not useful to specify an exact effective amount in advance.

The term “pharmaceutically acceptable salt” refers to those salts that are, within the scope of sound medical judgment, suitable for use in contact with the tissues of humans and lower mammals without undue toxicity, irritation, allergic response, and the like, and are commensurate with a reasonable benefit/risk ratio. Pharmaceutically acceptable salts are well-known in the art. They can be prepared in situ when finally isolating and purifying the compounds of the invention, or separately by reacting them with pharmaceutically acceptable non-toxic bases or acids, including inorganic or organic bases and inorganic or organic acids (Berge, 1977). The ‘free base’ form may be regenerated by contacting the salt with a base or acid, and isolating the parent compound in the conventional matter. The parent form of the compound differs from the various salt forms in certain physical properties, such as solubility in polar solvents, but otherwise the salts are equivalent to the parent form of the compound for the purposes of the present invention.

The term “treatment” as used herein refers to any treatment of a mammalian, for example human condition or disease, and includes: (1) inhibiting the disease or condition, i.e., arresting its development, (2) relieving the disease or condition, i.e., causing the condition to regress, or (3) stopping the symptoms of the disease.

The term ‘inhibit’ includes its generally accepted meaning which includes prohibiting, preventing, restraining, alleviating, ameliorating, and slowing, stopping or reversing progression, severity, or a resultant symptom. As such, the present method includes both medical therapeutic and/or prophylactic administration, as appropriate.

As used herein, the term “medical therapy” intendeds to include prophylactic, diagnostic and therapeutic regimens carried out in vivo or ex vivo on humans or other mammals.

‘Mammals’ include animals of economic importance such as bovine, ovine, and porcine animals, especially those that produce meat, as well as domestic animals, sports animals, zoo animals, and humans, the latter being preferred.

The term “subject” as used herein, refers to an animal, preferably a mammal, most preferably a human, who has been the object of treatment, observation or experiment.

As used herein ‘obesity’ refers to a condition whereby a person has a Body Mass Index (BMI), calculated as weight per height squared (kg/m²), of at least 30. Conventionally, those persons with a BMI of at least 25.9 to less than 30 are considered overweight. Conventionally, those persons with normal weight have a BMI of 19.9 to less than 25.9. The obesity herein may be due to any cause, whether genetic of environmental. Examples of disorders that may result in obesity or be the cause of obesity include overeating and bulimia, polycystic ovarian disease, craniopharyngioma, the Prader-Willi syndrome, Frohlich's syndrome, Type-II diabetes, GH-deficient subjects, normal variant short stature, Turners syndrome, and other pathological conditions showing reduced metabolic activity or a decrease in resting energy expenditure as a percentage of total fat-free mass, e.g. children with acute lymphoblastic leukemia.

### EXAMPLES

### Analytical Methods

Nuclear magnetic resonance spectra (¹H NMR and ¹³C NMR, APT) were determined in the indicated solvent using a Bruker Avance 500 (1H: 500 MHz, ¹³C: 125 MHz) at 300 K, unless indicated otherwise. The spectra were determined in deuterated dimethylsulfoxide obtained from Cambridge Isotope Laboratories Ltd. Chemical shifts (6) are given in ppm downfield from tetramethylsilane (1H, 13C). Coupling constants J are given in Hz. Peakshapes in the NMR spectra are indicated with the symbols ‘q’ (quartet), ‘dq’ (double quartet), ‘t’ (triplet), ‘dt’ (double triplet), ‘d’ (doublet), ‘dd’ (double doublet), ‘s’ (singlet), ‘bs’ (broad singlet) and ‘m’ (multiplet).

Melting points were recorded on a Büchi B-545 melting point apparatus.

Mass spectra were recorded on a Micromass QTOF-2 instrument with MassLynx software to acquire and reconstruct data. Exact masses were measured as quasimolecular ions [M+H]⁺.

All reactions involving moisture sensitive compounds or conditions were carried out under an anhydrous nitrogen atmosphere. All solvents were distilled freshly prior to use. All other commercially available chemicals were used without further purification.

Reactions were monitored by using thin-layer chromatography (TLC) on silica coated plastic sheets (Merck precoated silica gel 60 F254) with the indicated eluent. Spots were visualized by UV light (254 nm) or I₂.

### General Aspects of Syntheses

The specific compounds, of which the synthesis is described below, are intended to further illustrate the invention in further detail without restricting the scope of the invention in any way. Other embodiments of the invention will be apparent to those skilled in the art from consideration of the specification and practice of the invention disclosed herein. The specification and examples must be considered as illustrative only.

Scheme 1 outlines one synthesis of compounds of Formula I:

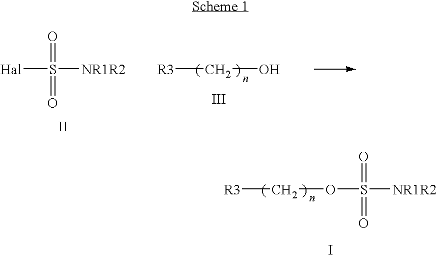

In a preferred embodiment, the process of scheme 1 is performed with compounds of formula II, wherein R1 and R2 are both hydrogen and wherein Hal is chloro.

In another preferred embodiment, the process of scheme I is performed with compounds of Formula III selected from the group consisting of: [(1S,2S,5S)-6,6-dimethylbicyclo[3.1.1]hept-2-yl]methanol; [(1R,2R,5R)-6,6-dimethylbicyclo[3.1.1]-hept-2-yl]methanol; [(1S,2S,5S)-6,6-dimethylbicyclo[3.1.1]hept-2-yl]ethanol, [(1S,2R,5S)-6,6-dimethylbicyclo[3.1.1]hept-2-yl]methanol, (1R,4S)-bicyclo[2.2.1]hept-2-yl-methanol, and (4S)-bicyclo[2.2.1]hept-5-en-2-ylmethanol, preferably [(1S,2S,5S)-6,6-dimethylbicyclo-[3.1.1]hept-2-yl]methanol.

The selection of the particular synthetic procedures depends on factors known to those skilled in the art such as the compatibility of functional groups with the reagents used, the possibility to use protecting groups, catalysts, activating and coupling reagents and the ultimate structural features present in the final compound being prepared.

Pharmaceutically acceptable salts may be obtained using procedures well-known in the art, for example by mixing a compound of the present invention with a suitable acid, for instance an inorganic acid or an organic acid.

### Example 1

### Synthesis of [(1S,2S,5S)-6,6-dimethylbicyclo[3.1.1]hept-2-yl]methyl-sulfamate—Compound 1

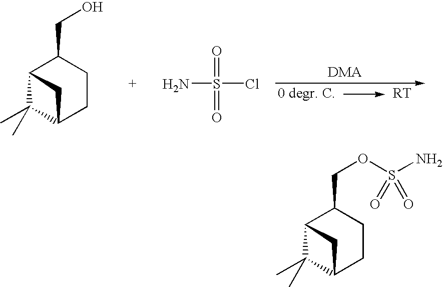

Sulfamoyl chloride (1.67 g, 14.45 mmol) was added in one portion to a stirred solution of [(1S,2S,5S)-6,6-dimethylbicyclo[3.1.1]hept-2-yl]methanol [(−)-trans-myrtanol] (1.115 g, 7.23 mmol) in absolute DMA (12 ml) at 0° C. The mixture was stirred at room temperature for 3 h and then poured into 50 ml of cold aqueous saturated sodium chloride solution (brine). The resulting solution was extracted with ethyl acetate (3×25 ml), the combined organic layers were washed with cold aqueous saturated sodium chloride solution (brine, 2×25 ml) and dried over MgSO₄. After concentration under reduced pressure, the crude product was purified by flash-chromatography on silica gel (ca. 45 g, eluent hexane:ethyl acetate=2:1) affording 1.558 g (6.68 mmol) pure sulfamate as a white solid; mp 67-68° C.; yield 92%.

¹H NMR (DMSO-d₆), δ: 0.82 (s, 3H, CH₃), 1.20 (s, 3H, CH₃), 1.20-1.38 (m, 2H), 1.53-1.63 (m, 1H), 1.69-1.79 (m, 2H), 1.81-1.87 (m, 2H), 2.01-2.08 (m, 1H), 2.23-2.33 (m, 1H), 3.80 (d, J=7.8 Hz, 2H, CHCH₂OSO₂), 7.38 (s, 2H, SO₂NH₂).

¹³C NMR (DMSO-d₆), δ: 17.23, 19.97, 22.88, 23.51, 26.44, 34.03, 38.74, 40.18, 41.51, 71.87. HR-MS (ESI, negative ion): found 232.1011; calcd. for C₁₀H₁₈NO₃S (M−H)⁻ 232.1007.

[α]_{D}²⁰-12.4° (c=0.525 mol/L in Methanol).

### Example 2

### Synthesis of [(1R,2R,5R)-6,6-dimethylbicyclo[3.1.1]hept-2-yl]methyl-sulfamate—Compound 2

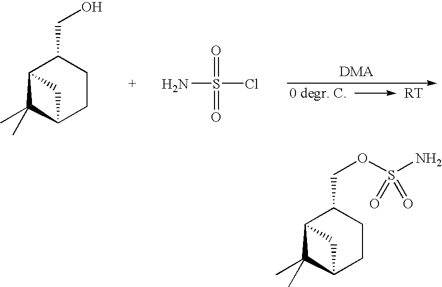

Sulfamoyl chloride (1.67 g, 14.45 mmol) was added in one portion to a stirred solution of [(1R,2R,5R)-6,6-dimethylbicyclo[3.1.1]hept-2-yl]methanol [(+)-trans-myrtanol] (1.115 g, 7.23 mmol) in absolute DMA (12 ml) at 0° C. The mixture was stirred at room temperature for 3 hours and then poured into 50 ml of cold aqueous saturated sodium chloride solution (brine). The resulting solution was extracted with ethyl acetate (3×25 ml), the combined organic layers were washed with cold aqueous saturated sodium chloride solution (brine, 2×25 ml) and dried over MgSO₄. After concentration under reduced pressure, the crude product was purified by flash-chromatography on silica gel (ca. 45 g, eluent hexane:ethyl acetate=2:1) affording 1.507 g (6.46 mmol) pure sulfamate as a white solid; mp 67-68° C.; yield 89%.

¹H NMR (DMSO-d₆), δ: 0.82 (s, 3H, CH₃), 1.20 (s, 3H, CH₃), 1.20-1.29 (m, 1H), 1.33 (d, J=9.9 Hz, 1H), 1.53-1.63 (m, 1H), 1.69-1.78 (m, 2H), 1.81-1.87 (m, 2H), 2.00-2.07 (m, 2H), 2.21-2.33 (m, 1H), 3.80 (d, J=6.5 Hz, 2H, CHCH₂OSO₂), 7.38 (s, 2H, SO₂NH₂).

¹³C NMR (DMSO-d₆), δ: 17.22, 19.96, 22.87, 23.50, 26.43, 34.02, 38.74, 40.17, 41.50, 71.86. HR-MS (ESI, negative ion): found 232.1012; calcd. for C₁₀H₁₈NO₃S (M−H)⁻ 232.1007.

[α]_{D}²⁰+12.8° (c=0.93 mol/L in Methanol).

### Example 3

### Synthesis of [(1S,2S,5S)-6,6-dimethylbicyclo[3.1.1]hept-2-yl]ethyl-sulfamate—Compound 3

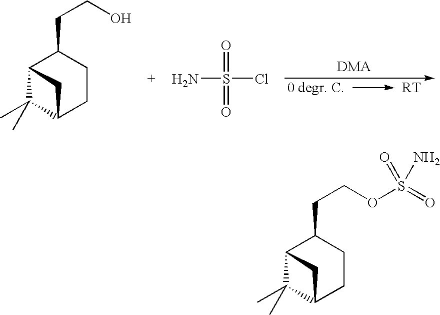

Sulfamoyl chloride (0.1156 g, 1 mmol) was added to a stirred solution of [(1S,2S,5S)-6,6-dimethylbicyclo[3.1.1]hept-2-yl]ethanol (0.084 g, 0.5 mmol) in absolute DMA (0.75 ml) at 0° C. The mixture was stirred at room temperature for 3 h and then poured into 10 ml of cold aqueous saturated sodium chloride solution (brine). The resulting solution was extracted with ethyl acetate (3×10 ml), the combined organic layers were washed with cold aqueous saturated sodium chloride solution (brine, 10 ml) and dried over MgSO₄. After concentration under reduced pressure, the crude product was purified by flash-chromatography on silica gel (ca. 3 g, eluent hexane:ethyl acetate=2:1) affording 0.221 g (0.445 mmol) pure sulfamate as a white solid; mp 49.5-51.5° C.; yield 89%.

¹H NMR (DMSO-d₆), δ: 0.80-0.92 (m, 1H), 0.99 (s, 3H, CH₃), 1.17 (s, 3H, CH₃), 1.40-1.53 (m, 1H), 1.63-2.13 (m, 8H), 2.23-2.40 (m, 1H), 4.01 (t, J=6.6 Hz, 2H, CH₂CH₂OSO₂), 7.36 (s, 2H, SO₂NH₂).

¹³C NMR (DMSO-d₆), δ: 21.30, 22.96, 25.90, 27.90, 32.95, 35.98, 36.60, 38.23, 40.74, 45.44, 67.72.

HR-MS (ESI, negative ion): found 246.1173; calcd. for C₁₁H₂₀NO₃S (M−H)⁻ 246.1164.

[α]D²⁰−19.3° (c=1.1, DCM).

### Examples 4 to 6

### Synthesis of [(1S,2R,5S)-6,6-dimethylbicyclo[3.1.1]hept-2-yl]methylsulfamate (Compound 4), (1R,4S)-bicyclo[2.2.1]hept-2-yl-methylsulfamate (Compound 5) and (4S)-bicyclo[2.2.1]hept-5-en-2-ylmethylsulfamate (Compound 6)

Compounds 4 to 6 can be prepared in a similar way be replacing the alcohol as starting material by any of [(1S,2R,5S)-6,6-dimethylbicyclo[3.1.1]hept-2-yl]methanol for the synthesis of compound 4, (1R,4S)-bicyclo[2.2.1]hept-2-yl-methanol for the synthesis of compound 5 and (4S)-bicyclo[2.2.1]hept-5-en-2-yl-methanol for the synthesis of compound 6, respectively.

### Example 7

### Formulation of Compound 1 Used in Animal Studies

For oral (p.o.) administration the compound was given as a suspension in a vehicle containing 1% methyl hydroxyethyl cellulose (m/m) and 0.1% wetting agent poloxamer 188 (nonionic polyoxyethylene-polyoxypropylene copolymer) in water. The preparation was done in a mortar with pestle and the pH adjusted to neutral condition.

For intraperitoneal (i.p.) administration: to the desired quantity of the solid compound 1 in a glass tube, some glass beads were added and the solid is milled by vortexing for 2 minutes. After addition of 1 ml of a solution of 1% methylcellulose and 5% mannitol in water, the compound is suspended by vortexing for 10 minutes. Finally the pH is adjusted to 7.

For intravenous (i.v.) administration: compound is dissolved in physiological saline (0.9% NaCl) and the pH was adjusted to 7.

### 7.1 Pharmacological Test Methods

The example numbers quoted in the pharmacological test methods relate to the preparation examples described below.

### 7.2 In Vitro Inhibition of Human Carbonic Anhydrase Isoenzymes

The test compounds of general Formula I in 96 well microplates were diluted with aqua bidest by using an automatic pipettor (CyBiWell®). From the different dilution plates, aliquots of 20 μl were transferred to the 96 well black assay plates with a pipetting station (Tecan Genesis®). In a second step, 148 μl of potassium phosphate buffer (20 mM, pH 7.4) was added, and as a third step, 20 μl of enzyme solution (1 μM human carbonic anhydrase isoenzyme I from erythrocytes (Sigma-Aldrich) or human carbonic anhydrase II from erythrocytes (Sigma Aldrich), or recombinant human carbonic anhydrase isoenzyme VB (R&D Systems), dissolved in potassium phosphate buffer) incubated for 60 min at room temperature and the fluorescence signal (Tecan Ultra® fluorescence reader; excitation wavelength: 280 nm; emission wavelength: 465 nm) read at the end of the preincubation period (FLU-1). After the preincubation time, 20 μl of aqueous dansylamide solution (1 mM dansylamide (Sigma-Aldrich), dissolved in hydrochloric acid) were added and the fluorescence signal read every 10 min for a period of 60 min at 37° C. For calculation, the fluorescence data of the time point 60 min (FLU-2) were used. The total volume of assay mixture amounted to 208 μl. The final concentration of carbonic anhydrase I was 10⁻⁷ M/L, the final concentration of carbonic anhydrase II was 10⁻⁷ M/L, the final concentration of carbonic anhydrase VB was 5×10⁻⁶ M/L, of dansylamide 10⁻⁷ or 2.5×10⁻⁶ or 5×10⁶, respectively and of compounds 10⁻⁷ M/L and 10⁻⁶ M/L. Final concentration of DMSO as compound solvent was 0.1%. Each microplate also contained blanks without compound and enzyme, controls without compound and ethoxzolamide (final concentration 5×10⁻⁸ M/L) as validation standard compound. All data reflect single measurements. Data were expressed as % inhibition after calculation by the formula:

$$\% inhibition = 100 \frac{( \begin{matrix}1 - FLU - {2}_{cpd} - FLU - {2}_{blank} - FLU - \\ {1}_{cpd} + FLU - {1}_{blank}\end{matrix} )}{( \begin{matrix}FLU - {2}_{control} - FLU - {2}_{blank} - FLU - \\ {1}_{control} - FLU - {1}_{blank}\end{matrix} )}$$

The % inhibition data for each compound and the respective final concentrations can be used for IC₅₀ calculations by using the Prism 4 software. Concentration action figures were calculated by applying the Prism algorithm for nonlinear regression (curve-fit): sigmoidal dose response with variable slope and the constraints: top: 100 and bottom 0.

In this test model, the test substances of general Formula I listed in Table 1 below showed the % inhibition data given below:

TABLE 1
hCA II inhibiting effect of the test substances in vitro

| Compound | | Final compound | | % |
|---|---|---|---|---|
| No. | Enzyme | concentration | | Inhibition |
| 1 | Carbonic anhydrase I | 0.1 | μM | 85 |
| 1 | Carbonic anhydrase II | 0.1 | μM | 56 |
| 1 | Carbonic anhydrase VB | 10 | μM | 70 |
| 2 | Carbonic anhydrase I | 0.1 | μM | 70 |
| 2 | Carbonic anhydrase II | 0.1 | μM | 33 |
| 2 | Carbonic anhydrase VB | 10 | μM | 75 |
| 3 | Carbonic anhydrase I | 0.1 | μM | 28 |
| 3 | Carbonic anhydrase II | 0.1 | μM | 15 |
| 3 | Carbonic anhydrase VB | 10 | μM | 55 |
| 4 | Carbonic anhydrase I | 1 | μM | 80 |
| 4 | Carbonic anhydrase II | 1 | μM | 72 |
| 4 | Carbonic anhydrase VB | 10 | μM | 53 |
| 5 | Carbonic anhydrase I | 1 | μM | 91 |
| 5 | Carbonic anhydrase II | 1 | μM | 77 |
| 5 | Carbonic anhydrase VB | 10 | μM | 41 |
| 6 | Carbonic anhydrase I | 1 | μM | 94 |
| 6 | Carbonic anhydrase II | 1 | μM | 76 |
| 6 | Carbonic anhydrase VB | 10 | μM | 40 |

### 7.3 Acute In Vivo Food Intake Test in Mice

The studies were carried out in male C57BI/6 mice (n=12 per group). The mice were kept on an inverted 12/12 h light/dark cycle (lights on 22:00). They were allowed food (high caloric diet) and water ad libitum. Food intake and water consumption was measured daily. The test compound of general Formula I was suspended in 1% methyl hydroxyethyl cellulose in water and 0.1% (v/v) of poloxamer 188 and administered by oral gavage at a dose of 50 mg/kg/day twice daily for 3 days. One half of the dose was administered at 8.00-10.00 h; the remaining half of the dose was administered between 14.00-15.00 h.

In the test model described above, the test substance caused a decrease of the animals' 72 hour food intake when compared to control as given in the following Table 2.

TABLE 2
Influence of test substances on food intake

| | food intake |
|---|---|
| Compound No. | [% of control] |
| 1 | 69.4% (day 3) |

7.4 Effect on Neurite Outgrowth from Hippocampal Neurons

Neurite outgrowth is an important parameter to evaluate the neurotrophic potency of a compound. The ability of compound 1 to increase neurite outgrowth was tested in cultures of embyronal hippocampal neurons. The hippocampal neurons from pregnant Wistar female rats were dissociated by trypsinization for 30 min at 37° C. (trypsin-EDTA, Gibco) in presence of DNAse I (Roche, Meylan). The reaction was stopped by addition of medium of Eagle modified by Dulbecco (DMEM; Gibco) with 10% of fetal bovine serum (Gibco). The suspension was triturated with a 10-ml pipette and using a 21G needle syringe and centrifuged at 350×g for 10 min at room temperature. The resulting pellet was re-suspended in culture medium containing Neurobasal medium (Gibco) with 2% of B27 supplement (Gibco) and 2 mM of glutamine (Gibco). Viable cells were counted in a Neubauer cytometer using the trypan blue exclusion test (Sigma) and seeded on the basis of 30 000 cells per Petri dish (Nunc) precoated with poly-L-lysine (Sigma). Cells were allowed to adhere 2 h and maintained in a humidified incubator at 37° C. in 5% CO2-95% air atmosphere.

After adhesion, vehicle and test compound at different concentrations (1 μM, 3 μM, 10 μM and 30 μM) were added to the medium. BDNF (50 ng/ml, 3.7 nM) was included as positive control for neurite growth. The test compound was tested in two independent cultures in parallel with control and BNDF cultures.

After the 3 days exposure of the neurons to the test compounds, cultures were washed in phosphate-buffered saline (PBS, Gibco) and fixed using 2.5% glutaraldehyde in PBS. Several pictures (˜80) of cells with neurites without any branching were taken per condition using a digital camera (Coolpix 995; Nikon) fixed on the microscope (Nikon, objective 40×). Neurites were outlined on computer screen using imaging software (Image-Pro Plus, France), which automatically calculates the length.

As expected 50 ng/ml BDNF treatment was associated with neurite sprouting from hippocampal neurons. In the first culture BNDF stimulated a mean increase in neurite length by 24.5 μm from 105.7±3.2 μm (mean±SEM; n=83) to 131.6±3.1 μm (n=79); in the second culture, the BNDF stimulated a mean increase in neurite length by 8.6 μm from 109.3±3.1 μm (mean±SEM; n=86) to 118.7±2.8 μm (n=82). When both cultures were normalized by the respective mean control neurite length there was a BNDF induced increase by 16.4%.

### Effect of Compound 1

As shown in table 3, all tested concentrations of compound 1 were associated with a significant increase in neurite length. The effect on neurite growth was comparable for all tested concentrations with increase of 9% to 15% as compared to the control level. The most effective concentration was 30 μM, which was comparable to the BDNF response.

TABLE 3
Effect of compound 1 on neurite outgrowth
compared to control condition

| | | mean length | |
|---|---|---|---|
| Group | N | (% of control) | SEM |
| Control | 169 | 100.0 | 2.0 |
| BDNF (1.85 nM) | 161 | 116.4 | 2.0 |
| Compound 1 (1 μM) | 171 | 110.9 | 2.3 |
| Compound 1 (3 μM) | 163 | 108.9 | 2.6 |
| Compound 1 (10 μM) | 176 | 113.2 | 2.2 |
| Compound 1 (30 μM) | 166 | 114.5 | 2.4 |

In conclusion, the present study indicates that compound 1 was effective at promoting neurite outgrowth in hippocampal neurons.

### 7.5 Electroconvulsive Shock Threshold Test (ECSTT) in the Mouse to Determine Anticonvulsive Properties of Tested Compound

The method, which detects pro-convulsant or anti-convulsant activity, follows that described by Swinyard et al. (J. Pharmacol. Exp. Ther. 1952, 106, 319-330). Mice were administered ECS (rectangular current, 0.4 s, 50 Hz) via corneal electrodes connected to a constant current shock generator (Ugo Basile: Type 7801).

Compound 1 was tested in a dose of 10, 30 and 100 mg/kg administered p.o. 60 minutes before ECS. A group wherein the vehicle is administered orally 60 minutes before the ECS serves a control. Diazepam (8 mg/kg p.o.), administered under the same experimental conditions, served as a positive anti-convulsive reference substance.

Treatment groups of 23 mice were exposed to ECS as follows: Animal no 1 was exposed to 30 mA of ECS. If animal no 1 did not convulse (tonic convulsions) within 5 seconds maximum, animal no 2 was exposed to a higher current of 40 mA. If there were also no convulsions in animal no 2, then the current was further increase in the following animals (increases of 10 mA) until the first tonic convulsion was observed. Once the first tonic convulsion was observed, the intensity of ECS was decreased by 5 mA for the next animal and then the intensity was decreased or increased by 5 mA from animal to animal depending on whether or the previous animal convulsed or not. The minimum intensity given was 25 mA and the maximum intensity given was 95 mA. The electroconvulsive shock threshold was determined as the mean current administered in the last 20 mice.

The results are represented as percent change from control. The number of deaths is also recorded approximately 30 minutes after the animal has been tested for convulsions. The test was performed blind. A positive percent change indicates an anticonvulsant effect. A negative percent change indicates a proconvulsant effect.

Quantitative data were analyzed by comparing treated groups with vehicle control using unpaired Student's t tests. Quantal data were analyzed by comparing treated groups with vehicle control using Fisher's Exact Probability tests.

TABLE 4
Effects of compound 1 and Diazepam in the electroconvulsive
shock (ECS) threshold test in the mouse (20 mice per group)

| | INTENSITY ADMINISTERED | | | |
|---|---|---|---|---|
| | (#) (mA) | | | |
| TREATMENT | | | % change | NUMBER OF |
| (mg/kg) | mean ± | | from | DEATHS |
| p.o. −60 min | s.e.m. | p value | control | AFTER ECS |
| Vehicle | 36.5 ± 1.0 | — | — | 1 |
| Compound 1 (10) | 40.0 ± 1.2* | 0.0334 | +10% | 2 |
| Compound 1 (30) | 43.5 ± 1.5*** | 0.0006 | +19% | 0 |
| Compound 1 | 88.0 ± 2.1*** | <0.0001 | +141% | 1 |
| (100) | | | | |
| Diazepam (8) | 81.8 ± 3.0*** | <0.0001 | +124% | 0 |

Student's t test: NS = Not Significant; *= p < 0.05; ***= p < 0.001.
Fisher's Exact test (number of death): no indication = not significant.
(#): minimum = 30 mA; maximum = 95 mA.

Compound 1 (10, 30 and 100 mg/kg) administered p.o. 60 minutes before the test dose-dependently and significantly increased the current threshold for inducing tonic convulsions at all 3 doses (+10%, p<0.05; +19%, p<0.001 and +141%, p<0.001, respectively). The maximal effect obtained at 100 mg/kg po was similar to the effect of the anti-convulsive reference compound diazepam. These data suggest that compound 1 has anti-epileptic properties.

### 7.6 Diabetic-Induced Neuropathic Pain Tests

In male Sprague Dawley rats (˜200 g) diabetes was induced by intravenous (tail vein) injection of a buffered solution of streptozotocin (STZ; Sigma, L'lsle d'Abeau Chesnes, France) at a dose of 55 mg/kg. STZ was prepared in 0.1 mol/l citrate buffer pH 4.5. Control group received an equivalent volume of citrate buffer. The day of STZ injection was considered as day 0. One week later, blood glucose level was monitored using a tail incision and a glucosimeter (Glucotrend, Roche Diagnostic GmbH, Germany). Rats with glycemia ≧260 mg/dl were deemed diabetic.

STZ-rats were distributed in 6 groups (n=10 animals each) in a way that the glycemia level was comparable between diabetic groups. The groups were:
1.) Non-diabetic control group treated with vehicle,
2.) STZ treated diabetes groups treated with vehicle,
3.)-5.) STZ treated diabetes groups treated with 10, 30 or 50 mg/kg p.o. of compound 1,
6.) STZ treated diabetes groups treated with the positive reference compound morphin (3 mg/kg sc).

Compound 1 and the vehicle (1% tylose suspension) were given p.o. 1 h before the behavioral assays. Morphine was injected subcutaneously 0.75 h before the behavioral assays. Two behavioral pain tests were carried out on day 10.

### Cold Bath Test on Day 10:

Each animal was placed on a cold platform (1-4° C.). The latency before the first reaction (licking, moving the paws, little leaps) was recorded with a maximal time of 30 s. A control animal can support a 20-30 s time before the first reaction in contrast with a STZ-rat which displays a reduced time (˜10 s) before the first reaction. This test serves as a measure for cold allodynia.

### Warm Plate (38° C.) Test on Day 10:

Animals were usually tested about 2-3 min after cold bath test. Each animal was placed into a glass cylinder on a warm plate (Slide warmer MH6616; Euromedex; France) adjusted to 38° C. The latency of the first reaction was recorded (licking, moving the paws, little leaps or a jump to escape the heat). Cut off time was set as 30 s. This test serves as a measure for warm allodynia.

Analysis of variance (ANOVA) was performed on data from each parameter. Fisher's Protected Least Significant Difference was used for pairwise comparisons: a p-value ≦0.05 were considered significant. The drug induced inhibition of the STZ-diabetes-induced allodynia and hyperalgesia was calculated by setting the respective response of the vehicle/control group as 100% and the STZ/Vehicle group as 0% inhibition.

% inhibition (compared to
STZ/vehicle group)

| TREATMENT (mg/kg) | cold bath test | warm plate test |
|---|---|---|
| STZ/Compound 1 (10) | 62* | 53 |
| STZ/Compound 1 (30) | 58* | 59* |
| STZ/Compound 1 (50) | 72* | 64* |

*significantly different (p < 0.05) from STZ/vehicle group

Compound 1 (10, 30 and 50 mg/kg) administered p.o. 1 hour before the test dose-dependently and significantly inhibited the STZ-diabetes-induced cold allodynia in the cold bath test at day 10. Compound 1 (30 and 50 mg/kg) administered p.o. 1 hour before the test dose-dependently and significantly inhibited the STZ-diabetes-induced warm allodynia in the warm plate test at day 10. These data suggest that compound 1 has potential in neuropathic pain, especially diabetic neuropathic pain.

### Example 8

### Pharmaceutical Preparations

For clinical use, compounds of Formula I are formulated into pharmaceutical compositions that are important and novel embodiments of the invention because they contain the compounds, more particularly specific compounds disclosed herein. Types of pharmaceutical compositions that may be used include, but are not limited to, tablet, pill, lozenge, dragee, troche, hard or soft capsule, powder, cachet, granule, suppository, solution, aqueous or oily suspension, emulsion, lotion, syrup, ointment, gel, paste, cream, foam, vapor, spray, aerosol or transdermal patch, and other types disclosed herein, or apparent to a person skilled in the art from the specification and general knowledge in the art. The active ingredient for instance, may also be in the form of an inclusion complex in cyclodextrins, their ethers or their esters. The compositions are used for oral, intravenous, subcutaneous, tracheal, bronchial, intranasal, pulmonary, transdermal, buccal, rectal, parenteral or other ways to administer. The pharmaceutical formulation contains at least one compound of Formula I in admixture with a pharmaceutically acceptable adjuvant, diluent and/or carrier. The total amount of active ingredients suitably is in the range of from about 0.1% (w/w) to about 100% (w/w) of the formulation, suitably from 0.5% to 50% (w/w) and preferably from 1% to 25% (w/w).

The compounds of the invention can be brought into forms suitable for administration by means of usual processes using auxiliary substances such as liquid or solid, powdered ingredients, such as the pharmaceutically customary liquid or solid fillers and extenders, solvents, emulsifiers, lubricants, flavorings, colorings and/or buffer substances. Frequently used auxiliary substances include magnesium carbonate, titanium dioxide, lactose, saccharose, sorbitol, mannitol and other sugars or sugar alcohols, talc, lactoprotein, gelatin, starch, amylopectin, cellulose and its derivatives, animal and vegetable oils such as fish liver oil, sunflower, groundnut or sesame oil, polyethylene glycol and solvents such as, for example, sterile water and mono- or polyhydric alcohols such as glycerol, as well as with disintegrating agents and lubricating agents such as magnesium stearate, calcium stearate, sodium stearyl fumarate and polyethylene glycol waxes. The mixture may then be processed into granules or pressed into tablets. A tablet is prepared using the ingredients below:

| Ingredient | Quantity (mg/tablet) |
|---|---|
| COMPOUND No. 1 | 10 |
| Cellulose, microcrystalline | 200 |
| Silicon dioxide, fumed | 10 |
| Stearic acid | 10 |
| Total | 230 |

The components are blended and compressed to form tablets each weighing 230 mg.

The active ingredients may be separately premixed with the other non-active ingredients, before being mixed to form a formulation. The active ingredients may also be mixed with each other, before being mixed with the non-active ingredients to form a formulation.

Soft gelatin capsules may be prepared with capsules containing a mixture of the active ingredients of the invention, vegetable oil, fat, or other suitable vehicle for soft gelatin capsules. Hard gelatin capsules may contain granules of the active ingredients. Hard gelatin capsules may also contain the active ingredients together with solid powdered ingredients such as lactose, saccharose, sorbitol, mannitol, potato starch, corn starch, amylopectin, cellulose derivatives or gelatin. Hard gelatin capsules can be prepared using the following ingredients:

| Ingredient | Quantity (mg/capsule) |
|---|---|
| COMPOUND No. 1 | 10 |
| Starch, dried | 95 |
| Magnesium stearate | 15 |
| Total | 120 |

The above ingredients are mixed and filled into hard gelatin capsules in 120 mg quantities.

Dosage units for rectal administration may be prepared (i) in the form of suppositories that contain the active substance mixed with a neutral fat base; (ii) in the form of a gelatin rectal capsule that contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration. Suppositories, each containing 1 mg of active ingredient, may be made as follows:

| Ingredient | Quantity (mg/suppository) |
|---|---|
| COMPOUND No. 1 | 20 |
| Saturated fatty acid glycerides | 2,000 |
| Total | 2,020 |

The active ingredient is passed through a appropriately sized mesh sieve and suspended in the saturated fatty acid glycerides previously melted using the minimum heat necessary. The mixture is then poured into a suppository mold of normal 2 g capacity and allowed to cool.

Liquid preparations may be prepared in the form of syrups, elixirs, concentrated drops or suspensions, e.g. solutions or suspensions containing the active ingredients and the remainder consisting, for example, of sugar or sugar alcohols and a mixture of ethanol, water, glycerol, propylene glycol and polyethylene glycol. An intravenous formulation may be prepared as follows:

| Ingredient | Quantity | |
|---|---|---|
| COMPOUND No. 1 | 1 | g |
| Arlatone G ™ | 100 | ml |
| EtOH | 100 | ml |
| Water, sterile | 800 | ml |

The compound is dissolved in the Arlatone G™, EtOH and water, and then the solution is slowly diluted with additional water.

If desired, such liquid preparations may contain coloring agents, flavoring agents, preservatives, saccharine and carboxymethyl cellulose or other thickening agents. Liquid preparations may also be prepared in the form of a dry powder, reconstituted with a suitable solvent prior to use. Solutions for parenteral administration may be prepared as a solution of a formulation of the invention in a pharmaceutically acceptable solvent. These solutions may also contain stabilizing ingredients, preservatives and/or buffering ingredients. Solutions for parenteral administration may also be prepared as a dry preparation, reconstituted with a suitable solvent before use.

By way of example and not of limitation, several pharmaceutical compositions are given, comprising preferred active compounds for systemic use or topical application. Other compounds of the invention or combinations thereof, may be used in place of (or in addition to) said compounds. The concentration of the active ingredient may be varied over a wide range as discussed herein. The amounts and types of ingredients that may be included are well known in the art.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A compound corresponding to Formula I 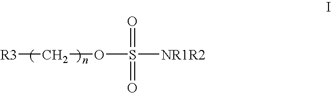
wherein
R1 and R2 are independently selected from the group consisting of hydrogen, alkyl, cycloalkyl, aryl and heteroaryl,
wherein said alkyl and cycloalkyl optionally may be substituted with at least one substitutent Y, and
wherein said aryl and heteroaryl optionally may be substituted with at least one substitutent Z; or
R1 and R2 form together with the nitrogen atom to which they are attached form a 5 or 6-membered ring which optionally may additionally contain 1 or 2 heteroatoms independently selected from the group consisting of: nitrogen, oxygen and sulfur, and wherein said 5 or 6-membered ring optionally may be substituted with at least one substituent Y;
R3 is selected from the group consisting of: (1S,2S,5S)-6,6-dimethyl-bicyclo[3.1.1]hept-2-yl; (1R,2R,5R)-6,6-dimethylbicyclo[3.1.1]hept-2-yl; (1S,2R,5S)-6,6-dimethyl-bicyclo-[3.1.1]hept-2-yl; (1R,4S)-bicyclo[2.2.1]hept-2-yl; (1S,4R)-3-methyl-bicyclo[2.2.1]hept-2-yl; bicyclo[2.2.2]oct-5-en-2-yl; (4S)-bicyclo[2.2.1]hept-5-en-2-yl; (1S,2R,4S)-1,7,7-trimethyl-bicyclo[2.2.1]hept-2-yl; (1R,2S,4R)-1,7,7-trimethyl-bicyclo[2.2.1]hept-2-yl; and (1R,2R,4R)-1,7,7-trimethylbicyclo[2.2.1]hept-2-yl;
n is an integer from 0 to 3;
Y is selected from the group consisting of: alkyl, alkoxy, thioalkyl, aryl, CO-aryl, heteroaryl, amino and carboxylalkyl; and
Z is selected from the group consisting of: alkyl, alkoxy, thioalkyl, halogen, aryl, CO-aryl, CN, heteroaryl and carboxylalkyl;
or a physiologically acceptable salt or solvate thereof.

2. A compound as claimed in claim 1, wherein R1 and R2 are independently selected from the group consisting of: hydrogen and C₁ to C₈ alkyl,
wherein said C₁ to C₈ alkyl optionally may be substituted with at least one substituent Y selected from the group consisting of: C₁-C₈ alkyl, C₁-C₈ alkoxy, C₁-C₈ thioalkyl, C₆-C₁₂aryl, CO—C₆-C₁₂aryl, C₆-C₁₂heteroaryl, amino, and carboxyl-C₁-C₈-alkyl.

3. A compound as claimed in claim 1, wherein R1 and R2 each represent hydrogen.

4. A compound as claimed in claim 1, wherein n is 1 or 2.

5. A compound as claimed in claim 4, wherein n is 1.

6. A compound as claimed in claim 1, selected from the group consisting of:
[(1S,2S,5S)-6,6-dimethylbicyclo[3.1.1]hept-2-yl]methyl-sulfamate,
[(1R,2R,5R)-6,6-dimethylbicyclo[3.1.1]hept-2-yl]methylsulfamate,
[(1S,2S,5S)-6,6-dimethylbicyclo[3.1.1]hept-2-yl]ethylsulfamate,
[(1S,2R,5S)-6,6-dimethylbicyclo[3.1.1]hept-2-yl]methylsulfamate,
(1R,4S)-bicyclo-[2.2.1]hept-2-yl-methylsulfamate, and
(4S)-bicyclo[2.2.1]hept-5-en-2-ylmethylsulfamate.

7. A compound as claimed in claim 6, wherein said compound is [(1S,2S,5S)-6,6-dimethylbicyclo[3.1.1]hept-2-yl]methylsulfamate.

8. A pharmaceutical composition comprising a compound as claimed in claim 1, and at least one pharmaceutically acceptable carrier or auxiliary substance.

9. A pharmaceutical composition as claimed in claim 8, wherein said composition is in the form of a tablet, pill, lozenge, dragee, troche, hard or soft capsule, powder, cachet, granule, suppository, solution, aqueous or oily suspension, emulsion, lotion, syrup, ointment, gel, paste, cream, foam, vapor, spray, aerosol or transdermal patch.

10. A method of treating or inhibiting a disorder or disease state selected from the group consisting of obesity, diabetes mellitus type I, diabetes mellitus type II, metabolic syndrome, syndrome X, diabetic neuropathy, diabetic retinopathy, diabetic nephropathy, diabetic microangiopathy, diabetic macroangiopathy, insulinoma, familial hyperinsulemic hypoglycemia, male pattern baldness, detrusor hyperreactivity, hypertension, dyslipoproteinaemia, hyperuricaemia, asthma, glucose metabolism disorders, Parkinsons disease, Alzheimers disease, angina, arrhythmia, coronary spasm, peripheral vascular disease, cerebral vasospasm, neurodegeneration, pain, impotence, glaucoma, bipolar disorders, migraine, alcohol dependence, cancer and cardiovascular diseases, or for effecting neuroprotection, analgesia, appetite regulation or cardioprotection, in a subject, said method comprising administering to said subject a therapeutically effective amount of a compound as claimed in claim 1.

11. A method as claimed in claim 10, wherein said disorder or disease state is arterial hypertension; hypertriglyceridaemia accompanied by dyslipoproteinaemia occurring with or without lowered HDL-cholesterol; a glucose metabolism disorder selected from the group consisting of insulin resistance, hyperglycemea and glucose intolerance; pain selected from the group consisting of neuropathic pain and chronic pain; or a cardiovascular disorder selected from the group consisting of cardioplegia, coronary heart disease, cerebrovascular diseases and peripheral occlusive arterial disease.

12. A process for preparing a compound corresponding to Formula I as claimed in claim 1, said process comprising:
reacting a compound corresponding to Formula II 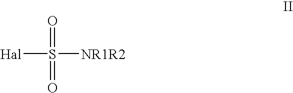
wherein Hal represents a halogen selected from the group consisting of: chloro and bromo,
with an alcohol corresponding to Formula III 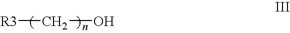
to yield a compound of Formula I.

13. The process as claimed in claim 12, wherein R1 and R2 each represent hydrogen and Hal represents chlorine.

14. The process as claimed in claim 12, wherein said compound of Formula III is selected from the group consisting of:
[(1S,2S,5S)-6,6-dimethyl-bicyclo[3.1.1]hept-2-yl]methanol;
[(1R,2R,5R)-6,6-dimethylbicyclo[3.1.1]hept-2-yl]methanol;
[(1S,2S,5S)-6,6-dimethylbicyclo[3.1.1]hept-2-yl]ethanol;
[(1S,2R,5S)-6,6-dimethylbicyclo[3.1.1]-hept-2-yl]methanol;
(1R,4S)-bicyclo[2.2.1]-hept-2-yl-methanol, and
(4S)-bicyclo[2.2.1]hept-5-en-2-ylmethanol.

15. The process as claimed in claim 14, wherein said compound of Formula III is [(1S,2S,5S)-6,6-dimethylbicyclo[3.1.1]hept-2-yl]methanol.
